(19) United States
(12) Patent Application Publication
(10) Pub. No.: US 2022/0387573 A1
(43) Pub. Date: Dec. 8, 2022

(54) COMBINATION THERAPY

(71) Applicant: ETHERNA IMMUNOTHERAPIES NV, Niel (BE)

(72) Inventors: Marina Cools, Niel (BE); Tim Van Assche, Niel (BE); Brenda De Keersmaecker, Niel (BE)

(73) Assignee: eTheRNA immunotherapies NV, Niel (BE)

(21) Appl. No.: 17/620,271

(22) PCT Filed: Jun. 29, 2020
(86) PCT No.: PCT/EP2020/068217

§ 371 Date: Dec. 17, 2021

(30) Foreign Application Priority Data
Jun. 27, 2019 (EP) 19182813.6

(51) Int. Cl. A61K 39/00; A61P 35/04

(52) U.S. Cl. CPC A61K 39/001156; A61K 39/001186; A61K 39/001188; A61K 39/001189; A61K 39/001192; A61P 35/04; A61K 2039/505; A61K 2039/55516; A61K 2039/876

(57) ABSTRACT

The present invention in general relates to combinations of mRNA molecules encoding CD40, caTLR4 and CD70 with mRNA molecules encoding tumor-associated antigens for use as therapeutic vaccine in the treatment of metastatic cancer patients primarily with stable malignant melanoma disease, but also extending into other cancer types and to patient whose disease has shown partial response on prior therapy. Said uses may further encompass the administration of checkpoint inhibitors. The present invention further provides administration schemes for such therapies focusing on administration of the therapeutic into lymph nodes, so called intra-nodal therapy.

### CROSS REFERENCES TO RELATED APPLICATIONS

This application is a national-stage application under 35 U.S.C. § 371 of International Application No. PCT/EP2020/068217, filed Jun. 29, 2020, which International Application claims benefit of priority to European Patent Application No. 19182813.6, filed Jun. 27, 2019.

### TECHNICAL FIELD

The present invention in general relates to combinations of mRNA molecules encoding CD40, caTLR4 and CD70 with mRNA molecules encoding tumor-associated antigens for use as therapeutic vaccine in the treatment of metastatic cancer patients primarily with stable malignant melanoma disease, but also extending into other cancer types and to patient whose disease has shown partial response on prior therapy. Said uses may further encompass the administration of checkpoint inhibitors. The present invention further provides administration schemes for such therapies focusing on administration of the therapeutic into lymph nodes, so called intra-nodal therapy.

### BACKGROUND

Melanoma is a malignant cancer that originates from the melanocytes and is the most dangerous form of skin cancer. Melanomas are typically found in the skin, but also occur in the mouth, intestines, or eyes. Over the past decades, the incidence of malignant melanoma has increased. According to the World Health Organization (WHO) about 132,000 new melanoma cases are diagnosed each year globally.

When melanoma is detected and treated at an early stage (resectable disease), wide local excision can be curative. However, 15-20% of melanoma patients develop metastases which may be loco regional or may reach a number of organs (typically lung, liver, and brain, and which can be unresectable and thus render a significantly worse prognosis.

In Europe, melanoma patients with resectable disease at high risk of relapse of their tumor after primary treatment had for a long time only limited adjuvant treatment options; observation or adjuvant therapy with interferon (IFN)α. However, the benefits of IFNα seem to be modest and the associated side effects might outweigh the benefit. New immunotherapies have been developed in the last decade, first for metastatic disease and nowadays also for the adjuvant treatment of melanoma patients.

The approvals of anti-cytotoxic T lymphocyte associated protein (CTLA)-4, anti-programmed cell death protein (PD)-1 and anti-programmed cell death protein ligand (PD-L)1 for first-line treatment of advanced (unresectable or metastatic) melanoma as well as the approval of B-Raf and mitogen-activated extracellular signal regulated kinase (MEK) inhibitors have opened new therapeutic options for metastatic patients. These therapies delivered unprecedented overall objective response rates, progression-free and overall survival to patients with unresectable or metastatic melanoma. The efficacy of monotherapy with anti-PD 1 antibodies (i.e. nivolumab and pembrolizumab) in melanoma can be substantially increased by the combination of ipilimumab and nivolumab. Yet, these impressive improvements came at the cost of toxicity and they are durable for only a minority of patients (typically less than 25%). Hence, alternative treatments with similar or better anti-tumor activity and a much better risk-benefit profile are highly desirable.

It was in particular an objective of the present invention to provide alternative treatments to enhance the treatment with anti-PD1 but in a tolerable way. It was found that this objective can be achieved by adding further immunostimulation, but via another modality and focusing on the dendritic cell priming against tumor associated antigens.

### SUMMARY

In a first aspect, the present invention provides a combination comprising:
one or more mRNA molecules encoding CD40L, caTLR4 and CD70; and
one or more mRNA molecules encoding a tumor-associated antigen (TAA);
for use in the treatment of metastatic cancer patients with stable disease as best objective response after first line checkpoint inhibitor therapy.

In a particular embodiment, of the present invention, said combination may be further combined with check point inhibitor therapy.

In a further embodiment of the present invention, said tumor-associated antigen is a melanoma antigen, preferably selected from the list comprising: tyrosinase, glycoprotein 100 (gp100), melanoma-associated antigen A3 (MAGE A3), melanoma-associated antigen C2 (MAGE C2) preferentially expressed antigen in melanoma (PRAME), and New York esophageal squamous cell carcinoma 1 (NY-ESO-1).

In yet a further embodiment of the present invention, said combination is for use in the treatment of metastatic cancer patients with stable disease as best objective response after at least 6 months of first line checkpoint inhibitor therapy.

In another specific embodiment, said checkpoint inhibitor therapy is selected from anti-PD-1 therapy and anti-CTLA4 therapy; preferably anti-PD-1 therapy, more in particular, an antagonistic antibody directed against PD-1, such as selected from the list comprising: nivolumab (BMS-936558/MDX1106), pidilizumab (CT-011) and pembrolizumab (MK-3475); preferably pembrolizumab (MK-3475).

In a specific embodiment of the present invention, said one or more mRNA molecules are formulated for parenteral administration; more in particular for intravenous, intratumoral, intradermal, subcutaneous, intraperitoneal, intramuscular or intranodal administration; preferably intranodal administration.

In a very specific embodiment, the present invention provides a combination comprising:
mRNA molecules encoding CD40L, caTLR4 and CD70; and
mRNA molecules encoding tyrosinase, glycoprotein 100 (gp100), melanoma-associated antigen A3 (MAGE A3), melanoma-associated antigen C2 (MAGE C2) and NY-ESO-1;
in combination with pembrolizumab;
for use in the treatment of a metastatic cancer patient by means of intranodal administration, wherein said patient is diagnosed with stable disease as best objective response after 6 months of first line pembrolizumab therapy.

In another very specific embodiment, the present invention provides a combination comprising:
mRNA molecules encoding CD40L, caTLR4 and CD70; and
mRNA molecules encoding tyrosinase, glycoprotein 100 (gp100), melanoma-associated antigen A3 (MAGE A3), melanoma-associated antigen C2 (MAGE C2) and preferentially expressed antigen in melanoma (PRAME);
in combination with pembrolizumab;
for use in the treatment of a metastatic cancer patient by means of intranodal administration, wherein said patient is diagnosed with stable disease as best objective response as best objective response after 6 months of first line pembrolizumab therapy.

In another particular embodiment, the combinations for use of the present invention may be administered according to the following administration scheme:
administrations 1-5: with an interval of about 1 week each;
administration 6: about 2 weeks after administration 5;
administration 7: about 3 weeks after administration 6;
administration 8: about 6 weeks after administration 7;
administration 9: about 6 weeks after administration 8.

In yet another particular embodiment, the combinations for use of the present invention may be administered according to the following administration scheme:
administrations 1-5: with an interval of about 1 week each;
administration 6: about 2 weeks after administration 5;
administration 7: about 3 weeks after administration 6;
administration 8: about 6 weeks after administration 7;
administration 9: about 6 weeks after administration 8;
and wherein said check point inhibitor therapy is administered at the same day of said combination prior, simultaneously or after administrations 1, 4, 6, 7, 8 and 9.

Where check point inhibitor therapy is used, it may further be administered between administrations 7 and 8; and between administrations 8 and 9.

The present invention also provides a combination for use as defined herein, wherein said combination comprises:
about 300 μg mRNA encoding CD40L;
about 300 μg mRNA encoding caTLR4;
about 300 μg mRNA encoding CD70;
about 900 μg mRNA encoding tumor-associated antigen(s)

In a very specific embodiment, the present invention provides a combination for use as defined herein, wherein said combination comprises:
about 600 μg mRNA encoding CD40L;
about 600 μg mRNA encoding caTLR4;
about 600 μg mRNA encoding CD70;
about 1800 μg mRNA encoding tumor-associated antigen(s).

### DETAILED DESCRIPTION

As already detailed herein above, the present invention provides a combination comprising:
one or more mRNA molecules encoding CD40L, caTLR4 and CD70; and
one or more mRNA molecules encoding a tumor-associated antigen (TAAs);
for use in the treatment of metastatic cancer patients with stable disease as best objective response after first line checkpoint inhibitor therapy.

Said combination of mRNA molecules encoding CD40L, caTLR4 and CD70, is herein also referred to as TriMix. In particular, TriMix is a mixture of messenger ribonucleic acids (mRNAs) that encodes potent dendritic cell (DC) and T-cell activation molecules, i.e. constitutively active Toll-like receptor 4 [caTLR4], human cluster of differentiation 40 ligand [CD40L] and T cell co-activation molecule [human CD70]. Essentially, TriMix represents a short-cut of the immune-activation circuit when expressed on DCs: caTLR4 confers a danger signal to DCs, CD40L delivers the retrograde signal of activated T-helper cells to DCs, and CD70 is a CD8⁺ T-cell activator secreted by activated DCs.

The relevance of each of these molecules in the context of the present invention is defined as follows:
caTLR4 (constitutively active Toll-like receptor 4): The binding of pathogen-associated molecular patterns to Toll-like receptors (TLRs) provides signals for DC maturation. Ligation of TLRs induces similar effects as CD40 ligation on DCs, namely, upregulation of co-stimulatory molecules and enhanced cytokine/chemokine secretion. Among the TLR ligands, lipopolysaccharide (LPS) (which binds to TLR4) is an attractive candidate because LPS-matured DCs have been shown to acquire an enhanced ability to stimulate specific T cells. The effect of LPS binding on DCs can be mimicked safely by engineering the DCs to express a constitutively active form of caTLR4; in this construct, the extracellular domain of the gene was deleted leading to constitutive activation of the truncated receptor (Bonehill et al., 2008).
CD40L (cluster of differentiation 40 ligand): CD40-CD40L interactions mediate one of the most potent DC activating signals. Commonly, CD40 ligation on DCs is provided by activated CD4⁺ T cells expressing CD40L. This process, which can be simulated by engineering DCs to express CD40L, leads to an upregulation of co-stimulatory molecules and enhanced production of cytokines/chemokines. In preclinical models, CD40 ligation increases the magnitude of CD4⁺ and CD8⁺ T-cell expansion. Especially for the induction of memory CD8⁺ T cells, it is believed that CD40 ligation is indispensable (Bonehill et al., 2008).
CD70 (cluster of differentiation 70): Like CD40L, CD70 is a member of the tumor necrosis factor (TNF) superfamily of co-stimulatory molecules. CD70 is the ligand of CD27, which is expressed on activated B cells, naive T cells, and on several subsets of memory T cells. CD27 ligation is a key contribution to the formation of effector and memory T-cell populations by promoting the survival and proliferation of primed T cells. Recently, CD70 has emerged as a key molecule for the priming of CD8⁺ T-cell responses. Indeed, several studies conducted in mice describe that CD40 ligation on DCs, whether in combination with a TLR ligand or not, induces CD70 expression by these DCs; and that the exponential expansion of CD8⁺ T cells after priming is almost completely dependent on the CD70/CD27 interaction (Bonehill et al., 2008).

In a further embodiment of the present invention, said tumor-associated antigen is a melanoma antigen, preferably selected from the list comprising: tyrosinase, glycoprotein 100 (gp100), melanoma-associated antigen A3 (MAGE A3), melanoma-associated antigen C2 (MAGE C2), preferentially expressed antigen in melanoma (PRAME) and New York esophageal squamous cell carcinoma 1 (NY-ESO-1). In a more preferred embodiment, the tumor-associated antigen is a combination of tumor-antigens, selected from the above list, even more preferred a combination of 5 antigens, being a combination of tyrosinase, glycoprotein 100 (gp100), melanoma-associated antigen A3 (MAGE A3), melanoma-associated antigen C2 (MAGE C2) and preferentially expressed antigen in melanoma (PRAME); or being a combination of tyrosinase, glycoprotein 100 (gp100), melanoma-associated antigen A3 (MAGE A3), melanoma-associated antigen C2 (MAGE C2) and New York esophageal squamous cell carcinoma 1 (NY-ESO-1).

The present invention is particularly directed to an immunotherapy against melanoma, which, besides messenger ribonucleic acid (mRNA) encoding 3 different immuno-stimulatory proteins (constitutively active Toll-like receptor 4 [caTLR4], cluster of differentiation 40 ligand [CD40L], and cluster of differentiation 70 [CD70]) also contains mRNA encoding tumor associated antigens (TAAs). The mixture of 3 mRNA's is designed to activate and ‘educate’ DCs in a structured manner to result in optimal stimulation of the immune system, both locally and systemically. The specific TAAs included are tyrosinase, glycoprotein 100 (gp100), melanoma-associated antigen (MAGE) A3, MAGE C2, preferentially expressed antigen in melanoma (PRAME) and/or New York esophageal squamous cell carcinoma 1 (NY-ESO-1) fused to an engineered version of dendritic cell (DC) lysosome-associated membrane protein (LAMP).

### Tumor Associated Antigens:

Tyrosinase:

The combination of the present invention can comprise the cancer antigen tyrosinase (Tyr), a fragment thereof, or a variant thereof. Tyrosinase is a copper-containing enzyme having tyrosine hydroxylase and dopa oxidase catalytic activities that can be found in microorganisms and plant and animal tissues. Specifically, tyrosinase catalyzes the production of melanin and other pigments by the oxidation of phenols such as tyrosine. Mutations in the TYR gene result in oculocutaneous albinism in mammals and non-pathological polymorphisms in the TYR gene contribute to variation in skin pigmentation. Additionally, in cancer or tumors such as melanoma, tyrosinase can become unregulated, resulting in increased melanin synthesis. Accordingly, tyrosinase is a cancer antigen associated with melanoma. In subjects suffering from melanoma, tyrosinase can be a target of cytotoxic T cell recognition.

Glycoprotein 100:

The combination of the present invention can comprise the cancer antigen Glycoprotein (gp100), a fragment thereof, or a variant thereof. Gp100 is also known as melanocyte protein PMEL and is a 661 amino acids long type I transmembrane glycoprotein, which is enriched in melanosomes. It is predominantly expressed in skin tissue, with limited expression found in e.g. liver, bone marrow, muscles, . . . .

MAGE A3:

The combination of the present invention can comprise the cancer antigen melanoma-associated antigen A3 (MAGE A3), a fragment thereof, or a variant thereof. The MAGE-A proteins are cancer testis antigens (CTA), which are expressed only in tumor cells and non-MHC expressing germ cells of the testis and placenta. MAGE-A proteins are expressed in a variety of human cancers including, but not limited to, melanoma, breast cancer, leukemia, thyroid cancer, gastric cancer, pancreatic cancer, liver cancer (e.g., hepatocellular carcinoma), lung cancer (e.g., non-small cell lung carcinoma), ovarian cancer, multiple myeloma, esophageal cancer, kidney cancer, head cancers (e.g., squamous cell carcinoma), neck cancers (e.g., squamous cell carcinoma), and urothelial cancer.

MAGE C2:

The combination of the present invention can comprise the cancer antigen melanoma-associated antigen C2 (MAGE C2), a fragment thereof, or a variant thereof. “MAGE-C2” is the melanoma antigen family C2. Among tumoral samples, MAGE-C2 is frequently expressed in seminomas, melanomas, and bladder carcinomas. It is also expressed in a significant fraction of head and neck carcinomas, breast carcinomas, non-small lung carcinomas and sarcomas.

PRAME:

The combination of the present invention can comprise the cancer antigen PRAME, a fragment thereof, or a variant thereof. PRAME, encoded by the PRAME gene, is a protein comprised of 509 amino acids and is expressed in testis, placenta, endometrium, ovary, adrenals, and in tissues derived from melanoma, lung, kidney, and head and neck carcinomas.

NY-ESO-1:

The combination of the present invention can comprise the cancer antigen New York-esophageal cancer-1 (NY-ESO-1; also called CTAG1), a fragment thereof, or a variant thereof. NY-ESO-1, encoded by the CTAG1B gene, is a 180 amino-acid long protein, with a glycine-rich N-terminal region and an extremely hydrophobic C-terminal region. NY-ESO-1 has restricted expression in normal tissues but frequent occurrence in cancer. NY-ESO-1 may be expressed in numerous cancers including, but not limited to, bladder, colorectal, esophagus, gastric, hepatocarcinoma, head and neck, melanoma, non-small cell lung, ovarian, pancreatic, synovial carcinoma and prostate cancers.

In a particular embodiment, the combination as defined herein is further combined with check point inhibitor therapy, such as selected from anti-PD-1 therapy and anti-CTLA4 therapy. Said checkpoint inhibitor therapy may encompass the use of a PD-1 inhibitor or a CTLA4 inhibitor.

As used herein, the term “PD-1 inhibitor” includes any compound able to directly or indirectly affect the regulation of PD-1 by reducing for example the expression of PD-1 (i.e., transcription and/or the translation) or its natural ligands PD-L1/PD-L2, or a PD-1 activity. It includes intracellular (e.g., agents that block a PD-1-associated signalling molecule or pathway, such as

SHP-1 and SHP-2) as well as extracellular PD-1 inhibitor. Without being so limited, such inhibitors include siRNA, antisense molecules, proteins, peptides, small molecules, antibodies, etc.

Hence, in a specific embodiment, said PD-1 pathway inhibitor is selected from the list comprising: a nanobody directed against PD-1, an antagonistic antibody directed against PD-1; a nanobody directed against PDL1, an antagonistic antibody directed against PDL1; or a derivative thereof. Derivatives of antibodies may for example include scFV, bispecific antibodies, . . . .

In an embodiment, the above-mentioned PD-1 inhibitor blocks/inhibits the interaction between PD-1 and a PD-1 ligand (e.g., PD-L1, PD-L2). Such inhibitor may target, for example, the IgV domain of PD-1 and/or PD-L1 and/or PD-L2, such as one or more of the residues involved in the interaction, as discussed above.

In an embodiment, the above-mentioned PD-1 inhibitor is a blocking antibody, such as an anti-PD-1 or anti-PD-L1/PD-L2 antibody. Blocking anti-PD-1 and/or anti-PD-L1/PD-L2 antibodies are well known in the art. Other blocking antibodies may be readily identified and prepared by the skilled person based on the known domain of interaction between PD-1 and PD-L1/PD-L2, as discussed above. For example, a peptide corresponding to the IgV region of PD-1 or PD-L1/PD-L2 (or to a portion of this region) could be used as an antigen to develop blocking antibodies using methods well known in the art.

By “anti-PD-1 antibody” or “anti-PD-L1” or “anti-PD-L2” in the present context is meant an antibody capable of detecting/recognizing (i.e. binding to) a PD-1, PD-L1 or PD-L2 protein or a PD-1, PD-L1 or PD-L2 protein fragment. In an embodiment, the above-mentioned antibody inhibits the biological activity of PD-1, such as PD-1-PD-L1/PD-L2 interaction or PD-1-mediated T cell inhibition. In another embodiment, the PD-1 or PD-L1/PD-L2 protein fragment is an extracellular domain of PD-1 or PD-L1/PD-L2 (e.g., the IgV domain).

In a very specific embodiment, said anti-PD-1 therapy, is an antagonistic antibody directed against PD-1, such as selected from the list comprising: nivolumab (BMS-936558/MDX1106), pidilizumab (CT-011) and pembrolizumab (MK-3475); preferably pembrolizumab (MK-3475). Alternatively the PD-L1 inhibitor atezolizumab may also be suitably used within the context of the invention.

As used herein, the term “CTLA4 pathway inhibitor” includes any compound which prevents or blocks CTLA4 initiated signaling. It may thus directly or indirectly affect the regulation of CTLA4 by reducing for example the expression of the CTLA4 receptor (i.e., transcription and/or the translation) or its natural ligands B7-1 (CD80) and B7-2 (CD86). Without being so limited, such inhibitors include siRNA, antisense molecules, proteins, peptides, small molecules, antibodies, nanobodies and derivatives of any of these. In a particular embodiment, said CTLA4 inhibitor may also be provided in the form of mRNA encoding said inhibitor, such as mRNA encoding an anti-CTLA4 antibody.

The preferred anti-CTLA4 antibody is a human antibody that specifically binds to human CTLA4. Human antibodies provide a substantial advantage in the treatment methods of the present invention, as they are expected to minimize the immunogenic and allergic responses that are associated with use of non-human antibodies in human patients.

Exemplary human anti-CTLA4 antibodies are described in detail in for example WO 00/37504. Such antibodies include, but are not limited to, 3.1.1, 4.1.1, 4.8.1, 4.10.2, 4.13.1, 4.14.3, 6.1.1, ticilimumab, 11.6.1, 11.7.1, 12.3.1.1, and 12.9.1.1, as well as tremelimumab and ipilimumab.

In yet a further embodiment of the present invention, said combination is for use in the treatment of metastatic cancer patients with stable disease as best objective response after at least 6 months (and preferably less than 12 months) of first line checkpoint inhibitor therapy. In the context of the present invention, “stable disease” is defined in accordance with RECIST1.1 (Schwartz et al., 2017) criteria as assessed on 2 consecutive imagings.

Briefly according to these criteria, stable disease is defined as: “Neither sufficient shrinkage (compared to baseline) to qualify for partial or complete response (CR or PR) nor sufficient increase (taking as reference the smallest sum of diameters at baseline or while on study, whichever is smallest) to qualify for progressive disease (PD)”. The classification of a response (either CR or PR) occurs in comparison to the sum of diameters at baseline, while progression is based on a comparison to the smallest of the sum of diameters at baseline or the smallest sum of diameters during the trial (nadir). Most protocols require the criteria for SD for a specified period (for example at least 4 weeks) before SD can be concluded. Thus, if imaging is conducted at 2 weeks and 4 weeks onstudy, and at 2 weeks the criteria for CR, PR or PD are not met, but at 4 weeks meets the criteria for PD, the best overall response is PD, not SD as the subject was not on-study long enough to qualify for SD.

In a specific embodiment of the present invention, said one or more mRNA molecules are formulated for parenteral administration; more in particular for intravenous, intratumoral, intradermal, subcutaneous, intraperitoneal, intramuscular or intranodal administration; preferably intranodal administration.

In a specific embodiment, said combination or mRNA molecules of the present invention is formulated for intranodal administration, such as in a suitable injection buffer, such as for example Ringer Lactate buffer.

For the direct in vivo administration of the mRNA, it is critical that the mRNA is delivered to the DCs. Since lymph nodes contain a relatively high number of DCs and are the primary site of immune induction (Van Lint et al., 2015), this is the most preferable approach. In murine models, it could be shown that this route of administration yielded similar or even better results than the injection of DCs electroporated with mRNA. For this reason, patients preferably receive direct administration of the mRNA into a lymph node (Van Lint et al., 2015).

In a very specific embodiment, the present invention provides a combination comprising:
mRNA molecules encoding CD40L, caTLR4 and CD70; and
mRNA molecules encoding tyrosinase, glycoprotein 100 (gp100), melanoma-associated antigen A3 (MAGE A3), melanoma-associated antigen C2 (MAGE C2) and preferentially expressed antigen in melanoma (PRAME);
in combination with pembrolizumab;
for use in the treatment of a metastatic cancer patient by means of intranodal administration, wherein said patient is diagnosed with stable disease as best objective response after 6 months of first line pembrolizumab therapy.

In another very specific embodiment, the present invention provides a combination comprising:
mRNA molecules encoding CD40L, caTLR4 and CD70; and
mRNA molecules encoding tyrosinase, glycoprotein 100 (gp100), melanoma-associated antigen A3 (MAGE A3), melanoma-associated antigen C2 (MAGE C2) and New York esophageal squamous cell carcinoma 1 (NY-ESO-1);
in combination with pembrolizumab;
for use in the treatment of a metastatic cancer patient by means of intranodal administration, wherein said patient is diagnosed with stable disease as best objective response after 6 months of first line pembrolizumab therapy.

In another particular embodiment, the combinations for use of the present invention may be administered according to the following administration scheme:
administrations 1-5: with an interval of about 1 week each;
administration 6: about 2 weeks after administration 5;
administration 7: about 3 weeks after administration 6;
administration 8: about 6 weeks after administration 7;
administration 9: about 6 weeks after administration 8.

For practical reasons, there may be some slight variation in the administration scheme, such as “about 1 week” is intended to be between 5-9 days (i.e. +/−2 days) after the previous administration. Similar variations may also occur for administrations which are 3-6 weeks apart.

In yet another particular embodiment, the combinations for use of the present invention may be administered according to the following administration scheme:
administrations 1-5: with an interval of about 1 week each;
administration 6: about 2 weeks after administration 5;
administration 7: about 3 weeks after administration 6;
administration 8: about 6 weeks after administration 7;
administration 9: about 6 weeks after administration 8;
and wherein said check point inhibitor therapy is administered at the same day of said combination prior, simultaneously or after administrations 1, 4, 6, 7, 8 and 9.

Where check point inhibitor therapy is used, it may further be administered between administrations 7 and 8; and between administrations 8 and 9.

In a preferred embodiment, the claimed combinations are administered on the same day as the checkpoint inhibitor, however, this may also be varied where needed.

The present invention also provides a combination for use as defined herein, wherein said combination comprises:
about 300 μg mRNA encoding CD40L;
about 300 μg mRNA encoding caTLR4;
about 300 μg mRNA encoding CD70;
about 900 μg mRNA encoding tumor-associated antigen(s)

In a very specific embodiment, the present invention provides a combination for use as defined herein, wherein said combination comprises:
about 600 μg mRNA encoding CD40L;
about 600 μg mRNA encoding caTLR4;
about 600 μg mRNA encoding CD70;
about 1800 μg mRNA encoding tumor-associated antigen(s).

### EXAMPLES

### Example 1

In this study, two different dosages of ECI-006 are administered intranodally on top of standard of care anti-PD1 (pembrolizumab or nivolumab) in advanced or metastatic melanoma patients with stable disease after 6 months of anti-PD1 therapy: Dose Level II (total dose 1800 μg mRNA) and Dose Level III (total dose 3600 μg mRNA).

### ECI-006

ECI-006 is an immunotherapy against melanoma, which besides messenger ribonucleic acid (mRNA) encoding 3 different immuno-stimulatory proteins (constitutively active Toll-like receptor 4 [caTLR4], cluster of differentiation 40 ligand [CD40L], and cluster of differentiation 70 [CD70]) also contains mRNA encoding tumor associated antigens (TAAs). The mixture of 3 mRNA's (EIA-001) is designed to activate and ‘educate’ DCs in a structured manner to result in optimal stimulation of the immune system, both locally and systemically. The specific TAAs included are tyrosinase, glycoprotein 100 (gp100), melanoma-associated antigen (MAGE) A3, MAGE C2, and preferentially expressed antigen in melanoma (PRAME) fused to an engineered version of dendritic cell (DC) lysosome-associated membrane protein (LAMP).

The mRNA molecules used herein are produced using the vector and procedures as described in WO2015071295, and thus further encompass a translation enhancer element and nuclear retention element as defined in WO2015071295.

The ECI-006 mRNA product is a parenteral solution in water for injection (WFI) without additives and is filled as single dose vials stored below −20° C. Prior to use the ECI-006 mRNA is reconstituted in a Hartmann buffer.

Immediately prior to use the vials are thawed at room temperature and reconstituted using a 1-mL syringe to add 880 μl Hartmann solution to the vial containing ECI-006. This results in a total volume of 1.1 mL with a volume concentration of 80% Hartmann. After reconstitution in Hartmann's, the mRNA should be used immediately from a microbiological point of view. However, if the reconstitution has taken place in controlled and validated aseptic conditions (responsibility of the pharmacy), the in-use storage time can be prolonged. The stability of the reconstituted drug product is validated up to 6 hours at 15-25° C.

### Anti-PD1

Pembrolizumab is administered to patients in line with the SmPC. Pembrolizumab (Keytruda®) is provided as 50 mg or 100 mg powder for concentrate for infusion in vials. After reconstitution, 1 mL of concentrate contains 25 mg of pembrolizumab.

Nivolumab is administered to patients in line with the SmPC. Nivolumab (Opdivo®) is provided as 40 mg, 100 mg or 240 mg powder for concentrate for infusion in vials. After reconstitution, 1 mL of concentrate contains 10 mg of nivolumab.

### Mode of Administration & Dosages

The injection solution is injected preferably into the inguinal lymph nodes of the patient under guidance of echography. The first injection is in a lymph node on the left or right side, the second one will alternate to a lymph node on the opposite site, i.e., right or left. The third injection will be done at again at the left or right side as done for the first injection, the next at the right or left side. The last injection will be on the opposite side. If inguinal lymph nodes are not available for injection, axillary nodes can be used at the discretion of the treating physician.

For Dose Level II, intranodal injection of ECI-006 in 500 μL injection buffer into the lymph nodes will be performed as described for Cohort 1. For Dose Level III, 1000 μL will be injected in 2 different lymph nodes, preferably on the same side (500 μL in each node).

Dose level II: contains 900 μg EIA-001 combined with 900 μg of TAAs (total dose of 1800 μg mRNA)

Dose level III: contains 1800 μg EIA-001 combined with 1800 μg of TAAs (total dose of 3600 μg mRNA)

Patients will receive 2 mg/kg or 400 mg pembrolizumab (Keytruda®) respectively as standard of care (cycles of 3 or 6 weeks over a period of 21 weeks).

Pembrolizumab is administered as an intravenous infusion over 30 minutes every 3 or 6 weeks. The recommended dose for melanoma patients is 2 mg/kg (every 3 weeks) or 400 mg (every 6 week regimen). Patients should be treated with pembrolizumab until disease progression or unacceptable toxicity. Dose modifications for pembrolizumab should be done based on the current clinical practice for patients receiving pembrolizumab.

Nivolumab is administered as an infusion of 240 mg every 2 weeks over 30 minutes intravenously or as an infusion of 480 mg every 4 weeks over 60 minutes intravenously.

### Treatment Scheme

Each patient receives a maximum of 9 administrations with ECI-006: the first 5 with an interval of 1 week each. The sixth administration is 2 weeks after the previous one. The seventh administration is 3 weeks after the previous one. The eighth and ninth administrations are 6 weeks after the previous one. Patients are randomized to the 2 different dose levels according to the following schedule:
At Dose Level II (1800 μg), an initial 3 patients are enrolled. Enrolment is staggered with at least 1 day between the first dose of each individual patient. The safety observation period consists of a period of 6 weeks after each patient's first treatment. The drug safety monitoring board (DSMB) will convene to complete an overall evaluation of the safety and tolerability after the third patient has completed the safety observation period.
If no Unmanageable Safety Issue (USI) occurs during safety observation period and the DSMB has given its recommendation, 3 patients are enrolled at Dose Level III (3600 μg). As before, the enrolment of these 3 patients is staggered with at least 1 day in between the first dosing of each patient. Again, the safety observation period for each patient consists of 6 weeks. The DSMB will complete an overall evaluation of safety and tolerability after three patients.
If no USI occurs during safety observation period in the 3 first patients randomized in the Dose Level III group and the DSMB has given its recommendation, an additional 4 patients may be randomized in the Dose Level III group of Cohort 2.
If 1 of the first 3 patients at Dose Level II or III experiences an USI in the safety observation period, 3 additional patients will be enrolled at the same Dose Level II or III, respectively, and evaluated as follows:
If no new USI is observed in the 3 additional patients at Dose Level II or III (total of 1 USI in 6 patients) during the first 6 weeks of treatment, the enrolment into Dose Level III Group can start or the enrolment of the 7th and last patient at Dose Level III can start, respectively.
If 1 or more USI are observed in the 3 additional patients (total of 2 or more USI in 6 patients) during the first 6 weeks of treatment, no dose escalation will be permitted. This dose will then be considered to have exceeded the Maximum Tolerated Dose (MTD) and the sponsor may decide to close the study when observed at Dose Level II or to amend the study for continuation at a lower dose level when observed at Dose Level III.

There is a total of maximum 148 days (21 weeks) between the first (Day 1) and the last administration of ECI-006 (Day 148). Patients receive a total of 9 administrations of ECI-006. Each patient receives 5 administrations of ECI-006 in a weekly interval (first 5 administrations) followed by 1 administration of ECI-006 2 weeks after the fifth dose, 1 administration of ECI-006 3 weeks after the sixth dose and 2 administrations of ECI-006 with a 6-week interval, resulting in a treatment duration of 21 weeks with a final activity assessment after 24 weeks. The first ECI-006 administration preferably occurs on the same day and prior to pembrolizumab treatment. If applicable later in the study, ECI-006 may be administered on the same day as and prior to pembrolizumab.

The detailed treatment schedule is thus as follows:
Day 1: start of treatment; baseline immune monitoring sampling, safety laboratory tests, first administration of ECI-006 and pembrolizumab
Day 8 (±2 days): second administration of ECI-006
Day 15 (±2 days): third administration of ECI-006
Day 22 (±2 days): fourth administration of ECI-006; administration of pembrolizumab (except if the patient is on a 6-weekly pembrolizumab regimen)
Day 29 (2 days): fifth administration of ECI-006; immune monitoring sampling
Day 43 (2 days): sixth administration ECI-006; administration of pembrolizumab; safety laboratory tests
Day 64 (±2 days): seventh administration ECI-006; administration of pembrolizumab (except if the patient is on a 6-weekly pembrolizumab regimen)
Day 71 (±2 days): immune monitoring sampling, safety laboratory tests, tumor biopsy (optional)
Day 85 (+2 days): first efficacy assessment; administration of pembrolizumab
Day 106 (+5 days): eight administration of ECI-006; administration of pembrolizumab (except if the patient is on a 6-weekly pembrolizumab regimen)
Day 127 (+5 days): Second efficacy assessment; administration of pembrolizumab; immune monitoring sampling, safety laboratory tests
Day 148 (+5 days): ninth administration of ECI-006; administration of pembrolizumab (except if the patient is on a 6-weekly pembrolizumab regimen)
Day 169 (±5 days): Third efficacy assessment, tumor biopsy (optional), safety laboratory tests, immune monitoring sampling, administration of pembrolizumab
Day 176 (±5 days): Follow-up Phone call (4 weeks after last administration of study medication)

### Patient Population

The patients to be treated using ECI-006 are defined as stage III or IV unresectable melanoma patients with stable disease after receiving first-line treatment with pembrolizumab for at least 6 months. These patients are very unlikely to show late partial or complete responses to pembrolizumab, but are as a standard kept on the anti-PD1 therapy, with the thought that the anti-PD1 does give some degree of resistance against progress, while it is recognized that these patients will most likely relapse in not a too distant future. Data is emerging suggesting that SD melanoma patient on aPD1 therapy have a higher level of immune activity and checkpoint inhibitor level than partial response or complete response patients, underscoring the potential for the use of a therapeutic vaccine to help tilt the immune battle to the advantage of the patient (Poster 2515 at ASCO 2019).

### Example 2: Clinical Data—Phase Ib

This example provides the tumor assessment protocols and data obtained in respect of patients being treated in accordance with the specifies of example 1.

Melanoma patients with stable disease according to the RECIST 1.1 criteria after 6 to 12 months of immunotherapy therapy evidenced by 2 consecutive imagings were eligible to participate to the study. The imagings to confirm stable disease are performed before the study, as part of the standard of care and using institutional guidelines. Next to melanoma patients with stable disease, also patients with a partial response after 6 to 12 months of immunotherapy deemed unlikely to respond further by the investigator were eligible to participate to the study.

At scheduled time points (i.e., Screening Visit and at Visits 10, 12 and 14) as well as upon suspicion of progression of disease, the investigator performs any additional investigations as needed to document the suspected progressive disease if this is in the best interest of the patient. Tumor assessment is done according to institutional guidelines or practice by the site radiology staff. Imaging is performed predose (no longer than 4 weeks before treatment with ECI-006) and 12 weeks (±2 days), 18 weeks (+5 days) and 24 weeks (+5 days) after the start of the treatment despite any potential treatment delays.

At the Screening Visit, tumor assessment is based on clinical, laboratory, and CT evaluations (preference)—and possibly MRI scan. In addition to chest, abdomen, pelvis and brain, all known sites of disease are assessed. All baseline assessments are performed as close as possible to the beginning of treatment, no longer than 4 weeks before treatment. Subsequent assessments include chest, abdomen and pelvis, and all known sites of disease and use the same imaging method as used at Baseline.

The golden-standard to assess the anti-tumor activity of radio- and chemo-therapies is the Response Evaluation Criteria in Solid Tumors (RECIST). During the development of cancer immunotherapies it quickly became apparent, however, that RECIST is not optimal. An increase in tumor size can be observed which is due to infiltration of immune cells (called “tumor ‘flare’” or “pseudoprogression”) preceding tumor regression. Using RECIST, this would be considered as progressive disease and the subsequent clinical response would be missed. Therefore, immune related criteria were developed taking “pseudoprogression” into account. Therefore, next to tumor assessment according to RECIST 1.1 (Eisenhauer et al. 2009), tumors will also be assessed according to iRECIST. iRECIST were developed to provide standardization for assessing response to immunotherapeutic agents on behalf of the RECIST Working Group (Seymour et al. 2017).

### Efficacy Assessment According to RECIST 1.1

Assessment of Overall Tumor Burden and Measurable Disease

In order to evaluate tumor response to therapy, it is essential to estimate the overall tumor burden at Baseline and use this as a comparator for subsequent measurements. Measurable disease is defined by the presence of at least one measurable tumor lesion. When CT scans have slice thickness greater than 5 mm, the minimum size for a measurable lesion should be twice the slice thickness.

At Baseline, tumor lesions/lymph nodes are categorized as measurable or non-measurable as follows.

Measurable Lesions

Measurable lesions must be accurately measured in at least one dimension (longest diameter in the plane of the measurement to be recorded) with a minimum size of:
10 mm by CT/MRI scan—(CT/MRI scan slice thickness no greater than 5 mm)
10 mm caliper measurement by clinical exam (lesions which cannot be accurately measured with calipers should be recorded as non-measurable)

Malignant lymph nodes: to be considered pathologically enlarged and measurable, a lymph node must be ≤15 mm in short axis when assessed by CT scan (CT scan slice thickness recommended to be no greater than 5 mm). At baseline and in follow-up, only the short axis is measured and followed.

Non-Measurable Lesions
All other lesions, including small lesions (longest diameter <10 mm or pathological lymph nodes with ≤10 to <15 mm short axis), as well as truly non-measurable lesions.
Lesions considered truly non-measurable include: leptomeningeal disease, ascites, pleural or pericardial effusion, inflammatory breast disease, lymphangitic involvement of skin or lung, abdominal masses/abdominal organomegaly identified by physical exam that is not measurable by reproducible imaging techniques.

### Specifications by Method of Measurement

Measurement of Lesions

All measurements are recorded in mm. All baseline evaluations are performed as close as possible to the treatment start and no longer than 28 days before start of treatment.

### Method of Assessment

The same method of assessment as well as the same technique is used to characterize each identified and reported lesion at Baseline and during follow-up. Imaging based evaluation is preferred over clinical examination, unless the lesion(s) being followed cannot be imaged but are assessable by a clinical exam.

CT/MRI Scan

CT/MRI is the preferred method to measure lesions selected for response assessment. Measurability of lesions on CT/MRI scan is based on the assumption that the CT/MRI slice thickness is ≤5 mm. When scans have a slice thickness 5 mm, the minimum size for a measurable lesion should be twice the slice thickness.

Chest X-Ray

Chest CT is preferred over chest x-ray, since CT is more sensitive than x-ray, particularly in identifying new lesions.

Clinical Lesions

Clinical lesions are only considered measurable when they are superficial and ≥10 mm diameter as assessed using calipers. For the case of skin lesions, documentation by color photography including a ruler to estimate the size of the lesion is suggested. As noted above, when lesions can be evaluated both by imaging and clinical exam, imaging evaluations should be undertaken.

Ultrasound

Ultrasound is not used as a method of measurement. If new lesions are identified by ultrasound during the conduct of the study, confirmation by CT or MRI is advised.

### Baseline Documentation of ‘Target’ and ‘Non-Target Lesions’

Target Lesions

When more than one measurable lesion is present at Baseline, all lesions up to a maximum of five lesions total (and a maximum of two lesions per organ) representative of all involved organs are identified as target lesions and will be recorded and measured at baseline.

Target lesions are selected based on their size (lesions with the longest diameter), be representative of all involved organs and should lend themselves to reproducible repeated measurements.

A sum of the diameters (SoD, longest for non-nodal lesions, short axis for nodal lesions) for all target lesions is calculated and reported as the baseline sum diameters. If lymph nodes are to be included in the sum, then as noted below, only the short axis is added to the sum. The baseline SoD is used as reference to further characterize any objective tumor regression in the measurable dimensions of the disease.

Lymph Nodes

Lymph nodes are normal anatomical structures which might be visible by imaging even if they are not involved by tumor. Pathological nodes which are defined as measurable and may be identified as target lesions must meet the criterion of a short axis of ≥15 mm by CT scan. Only the short axis of these nodes will contribute to the baseline sum. Nodes that have a short axis <10 mm are considered non-pathological and should not be recorded or followed.

Non-Target Lesions

All other lesions (or sites of disease) including pathological lymph nodes are identified as non-target lesions and also be recorded at Baseline. Measurements are not required and these lesions are followed as ‘present’, ‘absent’ or in rare cased ‘unequivocal progression’. In addition, it is possible to record multiple non-target lesions involving the same organ as a single item on the case report form.

### Tumor Response Evaluation According to RECIST 1.1

Evaluation of Target Lesions

Complete Response (CR): disappearance of all target lesions. Any pathological lymph nodes (whether target or non-target) must have a short axis <10 mm.

Partial Response (PR): at least a 30% reduction in the SoD of the target lesions compared to the baseline sum diameters.

Progressive Disease (PD): at least a 20% increase in the SoD of target lesions, taking as reference the smallest sum on study (this includes the baseline sum if that is the smallest on study). In addition to the relative increase of 20%, the sum must also demonstrate an absolute increase of at least 5 mm.

Note: the occurrence of one or more new lesions is also considered progression.

Stable Disease (SD): neither sufficient shrinkage to qualify for PR nor sufficient increase to qualify for PD, taking a reference the smallest sum of diameters while on study.

Target Lesions that Become ‘too Small to Measure’

All lesions (nodal and non-nodal) recorded at baseline have their actual measurements recorded at each subsequent evaluation, even then very small. If the radiologist is able to provide an actual measurement, even if it is below 5 mm, it is recorded.

Target Lesions that Split or Coalesce on Treatment

When non-nodal lesions ‘fragment’, the longest diameters of the fragmented portions is added together to calculate the target lesion sum.

As lesions coalesce, a plane between them maybe maintained that would aid in obtaining maximal diameter measurements of individual lesions. If the lesions have truly coalesced such that they are no longer separable, the vector of the longest diameter in this instance is the maximal longest diameter for the ‘coalesced lesion’.

Evaluation of Non-Target Lesions

While some non-target may actually be measurable they do not need to be measured and instead are only assessed qualitatively at the time points specified in the protocol.

CR: disappearance of all non-target lesions. All lymph nodes must be non-pathological in size (short axis <10 mm).

Non-CR/non-PD: persistence of one or more non-target lesion(s) above the normal limits.

PD: unequivocal progression of existing non-target lesions.

Note: the appearance of one or more new lesions is also considered progression.

Unequivocal Progression in Non-Target Disease

To achieve ‘unequivocal progression’ on the basis of non-target disease, there must be an overall level of substantial worsening in non-target disease such that, even in presence of SD or PR in target disease, the overall tumor burden has increased sufficiently to merit discontinuation of therapy.

A modest ‘increase’ in the size of one or more non-target lesions is usually not sufficient to qualify for unequivocal progression status.

### New Lesions

The appearance of new malignant lesions denotes disease progression. The finding of a new lesion should be unequivocal: i.e., not attributable to differences in scanning technique, change in imaging modality or findings thought to represent something other than tumor (e.g., some ‘new’ bone lesions might be simply healing or flare of preexisting lesions). This is particularly important then the patient's baseline lesions show PR or CR. For example, necrosis of a liver lesion may be reported on a CT scan as a ‘new’ cystic lesion, which it is not.

A lesion identified on a follow-up Visit in an anatomical location that was not scanned at baseline is considered a new lesion and will indicate disease progression. An example of this is the patient who has visceral disease at baseline and while in the study has a CR or MRI brain scan ordered which reveals metastases. The patient's brain metastases are considered to be evidence of PD even if he/she did not have brain imaging at baseline.

If a new lesion is equivocal, e.g., because of its small size, continued therapy and follow-up evaluation will clarify if it truly represents new disease. If repeat scans confirm there is definitely a new lesion, then progression should be declared using the date of the initial scan.

### Response Criteria (RECIST 1.1)

For patients who have measurable disease at baseline, Table 1 provides a summary of the overall response calculation at each time point.

TABLE 1
Time point response - patients with target (±non-target) disease

| Target lesions | Non-target lesions | New lesions | Overall response |
|---|---|---|---|
| CR | CR | No | CR |
| CR | Non-CR/non-PD | No | PR |
| CR | Not evaluated | No | PR |
| PR | Non-PD or not all | No | PR |
| | evaluated | | |
| SD | Non-PD or not all | No | SD |
| | evaluated | | |
| Not all | Non-PD | No | Not |
| evaluated | | | evaluable |
| PD | Any | Yes or No | PD |
| Any | PD | Yes or No | PD |
| Any | Any | Yes | PD |

Confirmation of Scans

Verification of response: confirmation of response is not required since it will not add value to the interpretation of study results per RECIST 1.1.

Verification of progression: progression of disease is verified in cases where progression is equivocal between 4 and 8 weeks after the first assessment of PD. If repeat scans confirm PD, then progression is declared using the date of the initial scan. If repeat scans do not confirm PD, then the patient is considered not to have PD per RECIST 1.1.

### Best Overall Response

The best overall response is determined once all the data for a patient are known. It is defined as the best response designation, as determined by the investigator, recorded between the data of start of treatment and the date of objectively documented progression per RECIST 1.1 or the date of subsequent therapy, whichever occurs first. For patients without documented progression or subsequent therapy, all available response designations will contribute to the BOR assessment. The patient's BOR assessment will depend on the findings of both target and non-target disease and will also take into consideration the appearance of new lesions.

Efficacy Assessment According to iRECIST

iRECIST are based on the classical RECIST modified to optimally assess anti-tumor activity of immunotherapies. The selection and measurement of the non-target and target lesions is identical to RECIST 1.1. The evaluation and management of new lesions is, however, different.

iRECIST Changes Versus RECIST

Management of New Lesions

In RECIST 1.1 new lesions only are recorded and not measured. The appearance of new lesions always means that the patient has progressive disease (PD). In iRECIST, new lesions are measured and followed up as the overall response depends on the evolution of new lesions and not only whether or not new lesions develop.

Hereto, new lesions are assessed using the RECIST 1.1 principles:
New lesion are classified as measurable or non-measurable.
Target new lesions (up to 5 and maximum 2 per site) are selected. Target new lesions are measured. The diameters of new lesions are not to be included in the sum of measurements of target lesions identified at baseline, but recorded as a sum of diameters of target new lesions.
Other new lesions (measurable/non-measurable) are recorded as non-target new lesions and reported as absent, present, increase, unequivocal increase per assessment timepoint.
New lesions do not have to resolve for subsequent stable disease or partial response providing that the next assessment did not confirm progression

Response Evaluation

iRECIST Response Terminology

Responses in analogy with RECIST 1.1 with prefix “i”:

For target lesions:
iCR/iPR: immune complete/partial response
iSD: immune stable disease
iUPD/iCPD: immune unconfirmed/confirmed progressive disease (see below)
For non-target lesions
iCR: immune complete response
non-iCR/non-iPD: persistence of one or more non-target lesion(s) above the normal limits.
iPD: immune progressive disease

### Time Point Response

In RECIST 1.1 once the patient is evaluated to have PD, he always remains to have PD. However, as indicated above, with immunotherapies pseudoprogression is possible which is followed by a partial or complete response. This response is being missed with RECIST 1.1. Therefore, at the first timepoint a patient is evaluated with PD according to RECIST 1.1, this is “unconfirmed” for iRECIST—termed iUPD (immune Unconfirmed Progressive Disease).

The iUPD must then be confirmed at the next tumor assessment (between 4 and 8 weeks after timepoint of iUPD assessment).

If confirmed, the patient has iCPD (immune Confirmed Progressive Disease).

If not confirmed, the patient can either stay iUPD or evolve to stable disease or partial or complete response.

This implies that the time point response is dynamic and is based on the change from baseline for iCR, iPR, iSD) or change from nadir for PD.

TABLE 2
Response criteria iRECIST

| | | | Overall response | |
|---|---|---|---|---|
| Target | Non-target | New | No prior | Prior |
| lesions | lesions | lesions | iUPD | iUPD |
| iCR | iCR | No | iCR | iCR |
| iCR | Non- | No | iPR | iPR |
| | iCR/non- | | | |
| | iPD | | | |
| iPR | Non- | No | iPR | iPR |
| | iCR/non- | | | |
| | iPD | | | |
| iSD | Non- | No | iSD | iSD |
| | iCR/non- | | | |
| | iPD | | | |
| iUPD with | iUPD with | Yes | NA | New lesions confirm iCPD if new lesions were |
| no change | no change | | | previously identified and increase in number or |
| OR | OR | | | size (≥5 mm in sum of diameters for target new |
| decrease | decrease | | | lesions or and increase for non-target new |
| from last | from last | | | lesions); If no change in new lesions (size or |
| timepoint | timepoint | | | number) from last timepoint, remains iUPD |
| iSD, iPR, | iUPD | No | iUPD | Remains iUPD unless iCPD confirmed based in |
| iCR | | | | further increase in size of non-target disease |
| | | | | (need not meet RECIST 1.1 criteria for |
| | | | | unequivocal PD) |
| iUPD | Non- | No | iUPD | Remains iUPD unless iCPD confirmed based on: |
| | iCR/Non | | | further increase in sum of diameters of at |
| | iUPD; iCR | | | least 5 mm, otherwise remains iUPD |
| iUPD | iUPD | No | iUPD | Remains iUPD unless iCPD confirmed based on |
| | | | | further increase in: |
| | | | | previously identified target lesion iUPD |
| | | | | in sum of diameter ≥5 mm and/or |
| | | | | Non-target lesion iUPD (prior |
| | | | | assessment - need not be |
| | | | | unequivocal PD) |
| iUPD | iUPD | Yes | iUPD | Remains iUPD unless iCPD confirmed based on |
| | | | | further increase in |
| | | | | previously identified target lesion iUPD |
| | | | | sum of diameter ≥5 mm and/or |
| | | | | previously identified non-target lesion |
| | | | | iUPD (need not be unequivocal) and/or |
| | | | | size or number of new lesions previously |
| | | | | identified |
| Non | Non- | Yes | iUPD | Remains iUPD unless iCPD confirmed based on |
| iUPD/PD | iUPD/PD | | | increase in size or number of new lesions |
| | | | | previously identified |

### RESULTS

A first patient has been enrolled in the study. The patient is 76 years of age and diagnosed as having Stage IV lung, gland and abdominal metastasized malign melanoma. The patient started in March 2019 in a combined immunotherapy based on ipilimumab and nivolumab, followed by a maintenance treatment with nivolumab monotherapy (240 mg every 2 weeks). From December 2019 up until May, 2020, the patient participated in the current study as defined in Example 1 at Dose Level II (9 administrations of 1800 μg each.

Radiological reevaluation of the patient confirmed stable disease, with moreover a 8.6% reduction of the target lesions, as evident from table 3:

TABLE 3
Size of the target lesions across the various visits

| | Screenign Visit | Visit 10 | EoS Visit |
|---|---|---|---|
| Target Lesion | 10 Dec. 2019 | 5 Mar. 2020 | 29 May 2020 |
| Lung Right | 12 mm | 11 mm | 09 mm |
| Lung Left | 13 mm | 13 mm | 12 mm |
| Lymph node | 25 mm | 23 mm | 23 mm |
| Adrenal gland | 22 mm | 22 mm | 22 mm |
| Total | 72 mm | 69 mm | 66 mm |

Moreover, the patient has made several statements confirming that the treatment has a positive impact on the patient's quality of life, such as ‘I have never felt so good’, ‘I feel fantastic’. Finally, the patient feels very vital as confirmed by the fact that the patient can make walks of up to 40 km, and can drive long distances (>1000 km) with the car without feeling tired.

Accordingly, these results confirm that the presently claimed combination is a valuable tool in the treatment of metastatic cancer patients with stable disease as best objective response after first line checkpoint inhibitor therapy.

### REFERENCES

Bonehill A et al., 2008. Enhancing the T-cell stimulatory capacity of human dendritic cells by co-electroporation with CD40L, CD70 and constitutively active TLR4 encoding mRNA. Mol Ther. 2008 June; 16(6):1170-80.
Eisenhauer E A, Therasse P, Bogaerts J, Schwartz L H, Sargent D, Ford R, et al. New response evaluation criteria in solid tumours: revised RECIST guideline (version 1.1). Eur J Cancer. 2009 January; 45(2):228-47.
Schwartz L H et al., 2016. RECIST 1.1—Update and Clarification: From the RECIST Committee. Eur. J. Cancer. 2016 July; 62:132-37.
Seymour L, Bogaerts J, Perrone A, Ford R, Schwartz L H, Mandrekar S, Lin N U, Litière S, Dancey J, Chen A, Hodi F S, Therasse P, Hoekstra O S, Shankar L K, Wolchok J D, Ballinger M, Caramella C, de Vries E G; RECIST working group. iRECIST: guidelines for response criteria for use in trials testing immunotherapeutics. 2017 March; 18(3):e143-el52
Van Lint S, Goyvaerts C, Maenhout S, Goethals L, Disy A, Benteyn D, et al. Preclinical evaluation of TriMix and antigen mRNA-based antitumor therapy. Cancer Res. 2012 Apr. 1; 72(7):1661-71.
Van Lint S, Renmans D, Broos K, Dewitte H, Lentacker I, Heirman C, et al. The ReNAissanCe of mRNA-based cancer therapy. Expert Rev Vaccines. 2015 February; 14(2):235-51.

1-14. (canceled)

15. A combination for treating a metastatic melanoma cancer patient with stable disease after a first line checkpoint inhibitor therapy, the combination comprising:
one or more mRNA molecules encoding CD40L, caTLR4, and CD70; and
one or more mRNA molecules encoding a melanoma tumor-associated antigen.

16. The combination of claim 15, wherein the melanoma tumor-associated antigen is selected from the group consisting of tyrosinase, glycoprotein 100 (gp100), melanoma-associated antigen A3 (MAGE A3), melanoma-associated antigen C2 (MAGE C2), preferentially expressed antigen in melanoma (PRAME), and New York esophageal squamous cell carcinoma 1 (NY-ESO-1).

17. The combination of claim 15, wherein the mRNA molecules of the combination are formulated for parenteral administration.

18. The combination of claim 15, wherein the mRNA molecules are formulated for intravenous administration, intratumoral administration, intradermal administration, subcutaneous administration, intraperitoneal administration, intramuscular administration, or intranodal administration.

19. The combination of claim 15, comprising:
about 300 μg mRNA encoding CD40L;
about 300 μg mRNA encoding caTLR4;
about 300 μg mRNA encoding CD70; and
about 900 μg mRNA encoding melanoma tumor-associated antigen(s).

20. The combination of claim 15, comprising:
about 600 μg mRNA encoding CD40L;
about 600 μg mRNA encoding caTLR4;
about 600 μg mRNA encoding CD70; and
about 1800 μg mRNA encoding melanoma tumor-associated antigen(s).

21. A method for treating a metastatic melanoma cancer patient with stable disease after a first line checkpoint inhibitor therapy, the method comprising:
administering to the patient after the first line checkpoint inhibitor therapy a therapeutically effective amount of the combination according to claim 15.

22. The method of claim 21, wherein the combination is administered after at least six months of the first line checkpoint inhibitor therapy.

23. The method of claim 21, wherein the first line checkpoint inhibitor therapy is selected from an anti-PD-1 therapy or an anti-CTLA4 therapy.

24. The method of claim 21, wherein the first line checkpoint inhibitor therapy is an anti-PD-1 therapy, the anti-PD-1 therapy being an antagonistic antibody directed against PD-1 and selected from the group consisting of nivolumab, pidilizumab, and pembrolizumab.

25. The method of claim 21, wherein the combination comprises:
about 300 μg mRNA encoding CD40L;
about 300 μg mRNA encoding caTLR4;
about 300 μg mRNA encoding CD70; and
about 900 μg mRNA encoding melanoma tumor-associated antigen(s)

26. The method of claim 21, wherein the combination comprises:
about 600 μg mRNA encoding CD40L;
about 600 μg mRNA encoding caTLR4;
about 600 μg mRNA encoding CD70;
about 1800 μg mRNA encoding melanoma tumor-associated antigen(s).

27. The method of claim 21, wherein the combination is administered according to an administration scheme comprising:
administrations 1-5 with an interval of about one week each;
administration 6 about two weeks after administration 5;
administration 7 about three weeks after administration 6;
administration 8 about six weeks after administration 7; and
administration 9 about six weeks after administration 8.

28. The method of claim 21, further comprising:
administering a checkpoint inhibitor to the patient in combination with administering the combination.

29. The method of claim 28, wherein:
the combination is administered according to an administration scheme comprising:
administrations 1-5 with an interval of about one week each;
administration 6 about two weeks after administration 5;
administration 7 about three weeks after administration 6;
administration 8 about six weeks after administration 7; and
administration 9 about six weeks after administration 8; and
the checkpoint inhibitor is administered on the same days of administrations 1, 4, 6, 7, 8, and 9 of the combination, the administrations of the checkpoint inhibitor occurring prior to the administration of the combination, simultaneously with the administration of the combination, or after the administration of the combination.

30. The method of claim 29, wherein the checkpoint inhibitor is additionally administered on a day between the days of administrations 7 and 8 of the combination and on a day between the days of administrations 8 and 9 of the combination.

31. The method of claim 28, wherein:
the melanoma tumor-associated antigen is selected from the group consisting of tyrosinase, glycoprotein 100 (gp100), melanoma-associated antigen A3 (MAGE A3), melanoma-associated antigen C2 (MAGE C2), and preferentially expressed antigen in melanoma (PRAME);
the checkpoint inhibitor is pembrolizumab;
the combination and the checkpoint inhibitor are administered by intranodal administration;
the first line checkpoint inhibitor therapy is a first line pembrolizumab therapy; and
the combination is administered after at least six months of the first line pembrolizumab therapy.

32. The method of claim 28, wherein:
the melanoma tumor-associated antigen is selected from the group consisting of tyrosinase, glycoprotein 100 (gp100), melanoma-associated antigen A3 (MAGE A3), melanoma-associated antigen C2 (MAGE C2), and New York esophageal squamous cell carcinoma 1 (NY-ESO-1);
the checkpoint inhibitor is pembrolizumab;
the combination and the checkpoint inhibitor are administered by intranodal administration;
the first line checkpoint inhibitor therapy is a first line pembrolizumab therapy; and
the combination is administered after at least six months of the first line pembrolizumab therapy.

33. The method of claim 28, wherein:
the melanoma tumor-associated antigen is selected from the group consisting of tyrosinase, glycoprotein 100 (gp100), melanoma-associated antigen A3 (MAGE A3), melanoma-associated antigen C2 (MAGE C2), and preferentially expressed antigen in melanoma (PRAME);
the checkpoint inhibitor is pembrolizumab;
the combination and the checkpoint inhibitor therapy are administered by intranodal administration;
the first line checkpoint inhibitor therapy is a first line nivolumab therapy; and
the combination is administered after at least six months of the first line nivolumab therapy.

34. The method of claim 28, wherein:
the melanoma tumor-associated antigen is selected from the group consisting of tyrosinase, glycoprotein 100 (gp100), melanoma-associated antigen A3 (MAGE A3), melanoma-associated antigen C2 (MAGE C2) and New York esophageal squamous cell carcinoma 1 (NY-ESO-1);
the checkpoint inhibitor is a pembrolizumab;
the combination and the checkpoint inhibitor therapy are administered by intranodal administration;
the first line checkpoint inhibitor therapy is a first line nivolumab therapy; and
the combination is administered after at least six months of the first line nivolumab therapy.
